(19) United States
(12) Patent Application Publication
(10) Pub. No.: US 2026/0250648 A1
(43) Pub. Date: Aug. 27, 2026

(54) LYSO-ORNITHINE LIPID BIOSURFACTANT OVERPRODUCTION SYSTEM

(71) Applicant: University of the Western Cape, Bellville, Cape Town (ZA)

(72) Inventors: Leonardo Joaquim VAN ZYL, Bellville, Cape Town (ZA); Marla TRINDADE, Bellville, Cape Town (ZA); Phillip VENTER, Bellville, Cape Town (ZA)

(73) Assignee: University of the Western Cape, Bellville, Cape Town (ZA)

(21) Appl. No.: 18/878,113

(22) PCT Filed: Jul. 28, 2023
(86) PCT No.: PCT/IB2023/057687

§ 371 Date: Dec. 23, 2024

(30) Foreign Application Priority Data
Jul. 29, 2022 (GB) 2211135.5

(51) Int. Cl. C12N 9/10; C07K 14/21; C12N 1/20; C12N 9/12; C12N 15/70; C12N 15/78; C12P 13/10; C12R 1/19; C12R 1/40

(52) U.S. Cl. CPC C12N 9/104; C07K 14/21; C12N 1/20; C12N 9/1205; C12N 15/70; C12N 15/78; C12P 13/10; C12N 2830/002; C12N 2830/34; C12R 2001/19; C12R 2001/40

(57) ABSTRACT

The present invention relates to a method for increasing production of a lyso-ornithine lipid in a microorganism, by expressing a heterologous ornithine acyl-ACP N-acyltransferase and elevating levels of an acyl carrier protein in the microorganism. Also provided for is a method of obtaining lyso-ornithine lipids with differing fatty acid chain lengths by varying the ornithine concentration. The invention further provides for recombinant microorganisms capable of producing increased levels of a lyso-ornithine lipid, which is useful as a biosurfactant. Also provided are lyso-ornithine lipids produced by the method or by the recombinant microorganism and application thereof as a biosurfactant.

### BACKGROUND OF THE INVENTION

The present invention relates to a method for increasing production of a lyso-ornithine lipid in a microorganism, comprising expressing a heterologous ornithine acyl-ACP N-acyltransferase and elevating levels of an acyl carrier protein in the microorganism. The invention also relates to recombinant microorganisms that are capable of increased heterologous production of a lyso-ornithine lipid and the use of the lyso-ornithine lipid as a biosurfactants. Further provided is a lyso-ornithine lipid produced by the methods or by the recombinant microorganisms, which has application as a biosurfactant.

Biosurfactants are biodegradable and environmentally compatible substitutes for petrochemically produced surfactants which maintain some of the highest production volumes amongst all synthetic chemicals. Their mild surfactant properties and wide operational capacity has identified them as suitable candidates to replace surfactants in certain applications whose economics suit their low production volumes. The main biosurfactant molecules mooted for industrial applications are sophorolipids, rhamnolipids and mannosylerythritol lipids, but the lack of diversity of biosurfactant structures is one of the problems requiring remedies if these molecules are to find wide-spread surfactant industry acceptance.

Identification of biosurfactants in the past relied completely on the culture-based approach. A typical biosurfactant screening study would use a minimal media supplemented with a plant or mineral oil as a carbon source. Only microorganisms that are able to mobilise the hydrophobic carbon substrates would be selected. The physical effect of the produced biosurfactant on the culture supernatant would then be measured by several well-established methods. While the culture-based approach has been successful at the identification of many useful secondary metabolites, it favours only microorganisms capable of being cultured in the media.

Previously, the inventors of the present invention used the functional metagenomic approach to identify a gene (olsB) encoding an ornithine acyl-ACP N-acyltransferase (OlsB) in Pseudomonas putida. This enzyme is an attractive target for biosurfactant production, but knowledge of its enzymatic characteristics, structure, and its interaction with its substrates is lacking.

Current methods of biosurfactant production for industrial use result in low yields of biosurfactant. In the specific case of lyso-ornithine lipid (LOL) biosurfactant production, to the inventors' knowledge, no solutions exist to increase yield of the LOL outside of the general large-scale production that is employed with all biosurfactants to increase yield and no method is known to change the type of biosurfactant produced. Current solutions to the problem of low yields include scaling up production, changing production conditions to optimize the yield and performing random mutagenesis to find overproduction strains. It is thus the aim of the present invention to improve the yield of lyso-orrnithine lipid biosurfactant production.

### SUMMARY OF THE INVENTION

The present invention relates to a method for increasing production of a lyso-ornithine lipid in a microorganism, by co-expressing a heterologous ornithine acyl-ACP N-acyltransferase and a heterologous acyl carrier protein in the microorganism. The invention further relates to a method of obtaining lyso-ornithine lipids with differing fatty acid chain lengths. Also provided are recombinant microorganisms capable of heterologously producing a lyso-ornithine lipid which has application as a biosurfactant. The invention further relates to a lyso-ornithine lipid produced by the method or by the recombinant microorganism and use thereof as a biosurfactant.

According to a first aspect of the present invention there is provided for a method for increasing production of a lyso-ornithine lipid in a microorganism, the method comprising:
i. introducing into the microorganism a nucleic acid encoding a heterologous ornithine acyl-ACP N-acyltransferase, wherein the nucleic acid encoding the heterologous ornithine acyl-ACP N-acyltransferase is contained on an expression vector;
ii. expressing the heterologous ornithine acyl-ACP N-acyltransferase in the microorganism; and
iii. elevating levels of an acyl carrier protein in the microorganism;
wherein expressing the heterologous ornithine acyl-ACP N-acyltransferase catalyses the synthesis of lyso-ornithine lipid and elevating the levels of the acyl carrier protein increases the production of lyso-ornithine lipid in the microorganism.

In a first embodiment of the method for increasing production of a lyso-ornithine lipid, elevating levels of the acyl carrier protein in the microorganism comprises introducing into the microorganism a nucleic acid encoding the acyl carrier protein contained on an expression vector, and expressing the acyl carrier protein.

According to a second embodiment of the method for increasing production of a lyso-ornithine lipid, the nucleic acid encoding the acyl carrier protein may be operably linked to a promoter, preferably an inducible promoter or a strong constitutive promoter.

According to a third embodiment of the method for increasing production of a lyso-ornithine lipid, the production of the lyso-ornithine lipid in the microorganism is increased relative to a microorganism that expresses the ornithine acyl-ACP N-acyltransferase and in which the levels of the acyl carrier protein have not been increased.

In a fourth embodiment of the method for increasing production of a lyso-ornithine lipid, the nucleic acid encoding the acyl carrier protein has a nucleotide sequence having at least 90% sequence identity with SEQ ID NO:4. For example, the first nucleic acid sequence may be about 95%, about 96%, about 97%, about 98%, about 99%, or 100% identical to the sequence of SEQ ID NO:4.

According to a fifth embodiment of the method for increasing production of a lyso-ornithine lipid, the production of the lyso-ornithine lipid in the microorganism is increased relative to a microorganism that expresses the ornithine acyl-ACP N-acyltransferase and in which the levels of the acyl carrier protein have not been increased.

In a further embodiment of the method for increasing production of a lyso-ornithine lipid, the ornithine acyl-ACP N-acyltransferase has an amino acid sequence substantially identical to SEQ ID NO:1.

According to another embodiment of the method for increasing production of a lyso-ornithine lipid, the first nucleic acid has a nucleotide sequence having at least 90% sequence identity with SEQ ID NO:2. For example, the first nucleic acid sequence may be about 95%, about 96%, about 97%, about 98%, about 99%, or 100% identical to the sequence of SEQ ID NO:2.

In a further embodiment of the method for increasing production of a lyso-ornithine lipid, the acyl carrier protein is a Pseudomonas putida acyl carrier protein or is derived from a Pseudomonas putida microorganism.

In another embodiment of the method for increasing production of a lyso-ornithine lipid, the acyl carrier protein has an amino acid sequence substantially identical to SEQ ID NO: 3.

In some embodiments of the method for increasing production of a lyso-ornithine lipid, the acyl carrier protein may be a heterologous acyl carrier protein that does not occur naturally in the microorganism.

In yet a further embodiment of the method for increasing production of a lyso-ornithine lipid, the microorganism in which the lyso-ornithine lipid is produced is Escherichia coli, Pseudomonas putida, Bacillus subtilis, Candida bombicola, Pichia stipitis or Saccharomyces cerevisiae. In particular, where the microorganism is Pseudomonas putida, a heterologous acyl carrier protein not obtained from Pseudomonas putida may be introduced into the microorganism, or alternatively expression of the native acyl carrier protein in Pseudomonas putida may be elevated. It will be appreciated by those of skill in the art that several methods of increasing expression of a native protein are known, including introducing an expression vector including a nucleic acid encoding the acyl carrier protein into the microorganism, including operably linked to a promoter such as an inducible promoter or a strong constitutive promoter. Alternatively other enhancers may be used to increase expression of the acyl carrier protein.

In some embodiments of the invention, the method may include stably transforming the microorganism with the nucleic acids encoding the ornithine acyl-ACP N-acyltransferase and/or the acyl carrier protein, for example using an integration vector.

According to a further embodiment of the method for increasing production of a lyso-ornithine lipid, the method may further comprise a step of culturing the microorganism in a medium containing ornithine. For example, the method may comprise a step of culturing the microorganism in a medium containing ornithine in the medium at a concentration from about 2 mM to about 200 mM.

In one embodiment of the method for increasing production of a lyso-ornithine lipid, changing the concentration of ornithine in the medium alters the length of the fatty acid chain of the lyso-ornithine lipid. In particular, a lower concentration of ornithine in the medium may result in lyso-ornithine lipids with a relatively longer fatty acid chain and a higher concentration of ornithine in the medium may result in lyso-ornithine lipids with a relatively shorter fatty acid chain. Thus, the invention further encompasses a method for obtaining lyso-ornithine lipids with differing fatty acid chain lengths.

In another embodiment of the method for increasing production of a lyso-ornithine lipid, the method may be carried out at a temperature of less than 35° C. For example, the method may be carried out at a temperature of about 25° C.

According to a further embodiment of the method for increasing production of a lyso-ornithine lipid, the method may be carried out in the presence of IPTG, to increase lyso-ornithine lipid production.

In yet a further embodiment of the method for increasing production of a lyso-ornithine lipid, the method may be carried out at a pH of between about pH 7 and about pH 8.

In an additional embodiment of the method for increasing production of a lyso-ornithine lipid, the method may further comprise a step of co-expressing a pantothenate kinase enzyme in the microorganism. In one embodiment, the pantothenate kinase enzyme may be obtained from or derived from E. coli, such as a pantothenate kinase enzyme having an amino acid sequence substantially identical to SEQ ID NO:6 and/or having a nucleotide sequence having at least 90% sequence identity with SEQ ID NO:5. For example, the pantothenate kinase enzyme may be encoded by a nucleic acid sequence about 95%, about 96%, about 97%, about 98%, about 99%, or 100% identical to the sequence of SEQ ID NO: 5.

In another embodiment of the method for increasing production of a lyso-ornithine lipid, the method may further comprise preparing a biosurfactant using the microorganism. In particular, the biosurfactant may be isolated and purified from the microorganism or the medium containing the microorganism.

According to a second aspect of the present invention, there is provided for a recombinant microorganism capable of expressing elevated levels of a lyso-ornithine lipid, wherein the microorganism comprises a first nucleic acid encoding a heterologous ornithine acyl-ACP N-acyltransferase and a second nucleic acid encoding an acyl carrier protein operably linked to an inducible promoter or a strong constitutive promoter, wherein the first and second nucleic acids are contained on at least one expression vector and wherein expressing the heterologous ornithine acyl-ACP N-acyltransferase catalyses the synthesis of lyso-ornithine lipid and expressing the acyl carrier protein increases the production of lyso-ornithine lipid.

According to a first embodiment of the recombinant microorganism of the invention, wherein the production of the lyso-ornithine lipid in the microorganism is increased relative to a microorganism that only comprises a nucleic acid encoding the ornithine acyl-ACP N-acyltransferase and not the nucleic acid encoding the acyl carrier protein operably linked to the inducible promoter or the strong constitutive promoter.

In a second embodiment of the recombinant microorganism of the invention, the ornithine acyl-ACP N-acyltransferase has an amino acid sequence substantially identical to SEQ ID NO:1.

According to a third embodiment of the recombinant microorganism of the invention, the first nucleic acid has a nucleotide sequence having at least 90% sequence identity with SEQ ID NO:2. For example, the first nucleic acid sequence may be about 95%, about 96%, about 97%, about 98%, about 99%, or 100% identical to the sequence of SEQ ID NO:2.

In another embodiment of the recombinant microorganism of the invention, the acyl carrier protein has an amino acid sequence substantially identical to SEQ ID NO:3.

In yet another embodiment of the recombinant microorganism of the invention, the second nucleic acid has a nucleotide sequence having at least 90% sequence identity with SEQ ID NO:4. For example, the first nucleic acid sequence may be about 95%, about 96%, about 97%, about 98%, about 99%, or 100% identical to the sequence of SEQ ID NO:4.

In a further embodiment of the recombinant microorganism of the invention, the acyl carrier protein is a Pseudomonas putida acyl carrier protein or is derived from a Pseudomonas putida microorganism.

In some embodiments of the recombinant microorganism of the invention, the acyl carrier protein may be a heterologous acyl carrier protein, that is not naturally expressed by the recombinant microorganism.

According to a further embodiment of the recombinant microorganism of the invention, the microorganism may further comprise a third nucleic acid encoding a pantothenate kinase enzyme, optionally operably linked to a promoter and/or contained on an expression vector, further wherein expressing the pantothenate kinase enzyme together with the heterologous ornithine acyl-ACP N-acyltransferase and the acyl carrier protein may further increase the production of lyso-ornithine lipid. In one embodiment, the pantothenate kinase enzyme may be obtained from or derived from E. coli, such as a pantothenate kinase enzyme having an amino acid sequence substantially identical to SEQ ID NO:6 and/or having a nucleotide sequence having at least 90% sequence identity with SEQ ID NO:5. For example, the pantothenate kinase enzyme may be encoded by a nucleic acid sequence about 95%, about 96%, about 97%, about 98%, about 99%, or 100% identical to the sequence of SEQ ID NO:5.

In some embodiments of the invention, the recombinant microorganism may be stably transformed with the nucleic acids encoding the ornithine acyl-ACP N-acyltransferase and/or the acyl carrier protein and/or the pantothenate kinase enzyme.

In yet another embodiment of the recombinant microorganism of the invention, the recombinant microorganism is a recombinant Escherichia coli bacterium or a Pseudomonas putida bacterium.

According to a third aspect of the present invention there is provided for a lyso-ornithine lipid produced by the method for increasing production of a lyso-ornithine lipid described herein or by the recombinant microorganism described herein.

In a fourth aspect of the present invention there is provided for use of the lyso-ornithine lipid as described herein as a biosurfactant. The biosurfactant may be used as an emulsifier, and the lyso-ornithine lipid preferably has an emulsification capacity after 24 hours (EC₂₄) of at least about 60%, preferably at least about 65%, more preferably at least about 70%, when emulsifying in paraffin.

### BRIEF DESCRIPTION OF THE FIGURES

Non-limiting embodiments of the invention will now be described by way of example only and with reference to the following figures:

FIG. 1: Ornithine lipid biosynthesis in bacteria. In reaction one OlsB catalyses the formation of lyso-ornithine lipid (LOL) by utilizing ornithine and a 3-hydroxyacyl-AcpP. In reaction two OlsA catalyses the formation of ornithine lipid (OL) by utilizing LOL and an acyl-AcpP. This form of ornithine lipid is designated the S1 form. The length and saturation of the fatty acids involved can vary. OlsB: ornithine acyl-ACP N-acyltransferase. OlsA: lyso-ornithine lipid O-acyltransferase. AcpP: acyl carrier protein.

FIG. 2: Enzymatic pathway illustrating the origin of the 3-hydroxyacyl-AcpP substrates for OlsB in vivo and invitro. The pathway indicated with solid black arrows illustrates fatty acid biosynthesis that produces the 3-hydroxyacyl-AcpP substrates in vivo. The pathway indicated with a dashed black arrow shows the direct acylation of holo-AcpP by an acyl-AcpP synthetase (AasS) in vitro.

FIG. 3: A diagram of the pETDuet-1/olsB vector used in cell line B.

FIG. 4: A diagram of the pCDFDuet-1/acpP vector used in cell line B.

FIG. 5: Emulsification assay testing the presence of biosurfactants in the supernatants of the produced cell lines. A: Control cell line containing pETDuet-1/olsB and an empty pCDFDuet-1 vector. B: Cell line containing pETDuet-1/olsB and pCDFDuet-1/acpP. C: An image insert of the control cell line containing empty pETDuet-1 and pCDFDuet vectors.

FIG. 6: Thin layer chromatography of the supernatants of the produced cell lines. A: Control cell line containing pETDuet-1/olsB and an empty pCDFDuet-1 vector. B: Cell line containing pETDuet-1/olsB and pCDFDuet-1/acpP. C: An image insert of control cell line containing empty pETDuet-1 and pCDFDuet vectors.

FIG. 7: HPLC chromatogram from the C18 reverse phase chromatography of the supernatants of lyso-ornithine lipid overexpression cell line B cultured with 20 mM or 200 mM ornithine supplementation. The control cell line is cell line Z. Eight peaks, visible in the lyso-ornithine lipid over-expression cell lines, but not in the control cell line are indicated with arrows.

FIG. 8: Clustered column chart showing the peak areas of the specific lyso-ornithine lipids produced in cell line A and cell line B. The average peak areas are displayed, and the standard deviation is indicated with error bars.

FIG. 9: Clustered column chart of the summed peak areas of the lyso-ornithine lipids produced in cell line A and cell line B. The average summed peak areas are displayed, and the standard deviation is indicated with error bars.

FIG. 10: Nucleotide sequence of the olsB gene (SEQ ID NO:1).

FIG. 11: Amino acid sequence of ornithine acyl-ACP N-acyltransferase (SEQ ID NO: 2).

FIG. 12: Nucleotide sequence of the acpP gene (SEQ ID NO:3).

FIG. 13: Amino acid sequence of acyl carrier protein (SEQ ID NO:4).

FIG. 14: A graph showing the effect of co-expression of E. coli panK on LOL production. The Y axis represents the peak area under the curve. The X axis represent the four fatty acid chain lengths (congeners) produced by strain “B” and strain “G”, without (−) and with (+) pantothenic acid (PA) supplementation.

FIG. 15: A graph of the peak areas of the four LOL fatty acid chain lengths (congeners) produced in cell line “B” at 25° C., 30° C. and 37° C. LOL production was quantified by HPLC.

FIG. 16: A graph of the peak areas of the four LOL fatty acid chain lengths (congeners) produced in cell lines “A”, “B”, “C”, “D”, “E” (negative control), “F” and “Z” (negative control) at 25° C. and 35° C. LOL production was quantified by HPLC.

FIG. 17: A graph of the peak areas of the LOL fatty acid chain lengths (congeners) produced in cell line “B” at different IPTG inducer concentrations, as determined through HPLC quantification.

FIG. 18: A graph pf the peak areas of the LOL fatty acid chain lengths (congeners) produced in cell line “B” at different medium pH values determined through HPLC quantification.

### SEQUENCE LISTING

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. The accompanying sequence listing is hereby incorporated by reference in its entirety.
SEQ ID NO:1—Nucleotide sequence of the olsB gene
SEQ ID NO:2—Amino acid sequence of ornithine acyl-ACP N-acyltransferase
SEQ ID NO:3—Nucleotide sequence of the acpP gene
SEQ ID NO:4—Amino acid sequence of acyl carrier protein
SEQ ID NO:5—Nucleotide sequence of the panK gene
SEQ ID NO:6—Amino acid sequence of the pantothenate kinase enzyme

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

The teachings of all patents, published applications and references cited herein are incorporated by reference in their entirety.

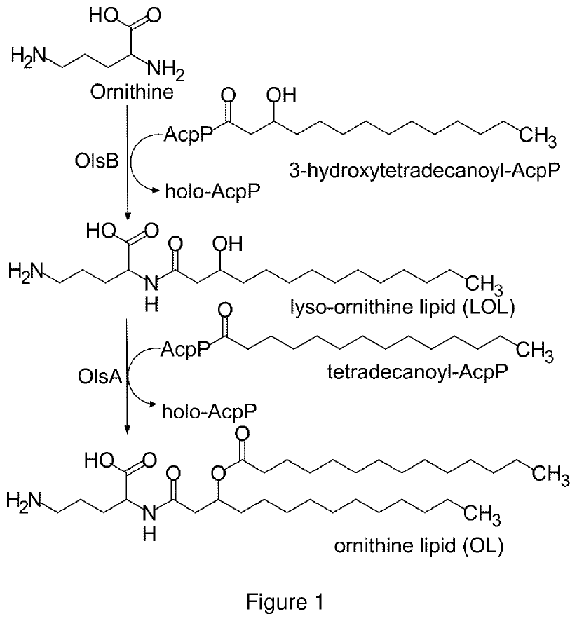

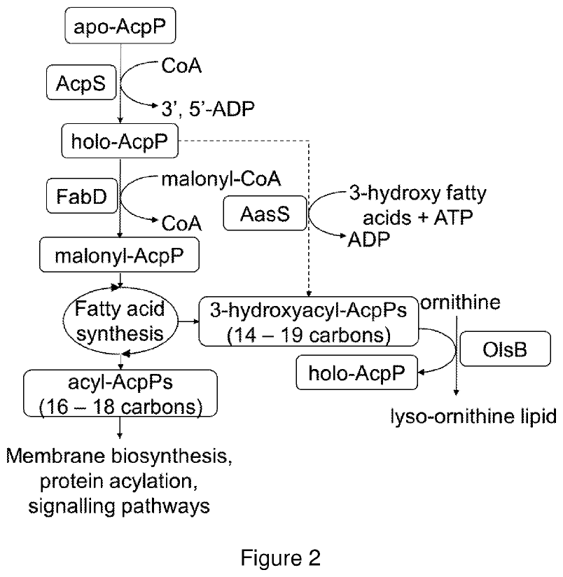

WO 2018/055563 A1 describes a gene encoding a lyso-ornithine lipid producing enzyme, ornithine acyl-ACP N-acyltransferase, isolated from Pseudomonas putida, namely the olsB gene. The ornithine acyl-ACP N-acyltransferase has been shown to catalyse the formation of lyso-ornithine lipid (LOL) by utilizing ornithine and a 3-hydroxyacyl-AcpP in a first reaction. In a second reaction, OlsA catalyses the formation of ornithine lipid (OL) by utilizing LOL and an acyl-AcpP (FIG. 1). Various enzymes or cofactor proteins involved in the LOL production pathway were co-expressed with the olsB gene, including AcpP, AcpS, AasS (FIG. 2). However, only co-expression with AcpP was found to increase LOL production. The inventors of the present invention have thus ascertained the structure of the acyl carrier protein from Pseudomonas putida, the cofactor protein of the ornithine acyl-ACP N-acyltransferase, through x-ray crystallography, to investigate the interaction of these two peptides.

The inventors of the present invention have further developed an enzyme co-expression system to produce a lyso-ornithine lipid overproduction strain. In addition, lyso-ornithine lipid quantification and purification methods were developed.

The ornithine acyl-ACP N-acyltransferase was found to have high substrate specificity for ornithine but could vary in the length of the fatty acid moiety attached to ornithine. Two novel lyso-ornithine lipids, containing shorter fatty acids, were identified. The co-expression of the acyl carrier protein and the ornithine acyl-ACP N-acyltransferase led to significantly higher lyso-ornithine lipid production. In addition, preliminary results for co-expression of the E. coli panK gene, with the acyl carrier protein and the ornithine acyl-ACP N-acyltransferase, also showed increased yield of lyso-ornithine lipids. Finally, various expression parameters were varied in order to optimise the lyso-ornithine lipid production method to arrive at optimum temperature and pH conditions for the production of lyso-ornithine lipid, as well as induction with IPTG, which also yielded promising results.

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

The terms “nucleic acid”, “nucleic acid molecule” and “polynucleotide” are used herein interchangeably and encompass both ribonucleotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides.

The term “heterologous” as used herein with reference to the ornithine acyl-ACP N-acyltransferase and/or the acyl carrier protein refers to an ornithine acyl-ACP N-acyltransferase and/or an acyl carrier protein that does not occur naturally in the microorganism. A heterologous ornithine acyl-ACP N-acyltransferase and/or heterologous acyl carrier protein may thus include an ornithine acyl-ACP N-acyltransferase and/or an acyl carrier protein from other bacteria or organisms. The heterologous ornithine acyl-ACP N-acyltransferase and/or the heterologous acyl carrier protein of the invention are intended for expression in a bacterial cell or a yeast cell expression system according to a non-limiting embodiment of the present invention. Suitable bacterial or yeast microorganisms for increased production of lyso-ornithine lipid of the present invention may include but are not limited to: Escherichia coli, Pseudomonas putida, Bacillus subtilis, Candida bombicola, Pichia stipitis or Saccharomyces cerevisiae.

The term “isolated”, is used herein and means having been removed from its natural environment.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

In some embodiments, nucleic acid constructs of the invention may include, without limitation, nucleotide sequences encoding ornithine acyl-ACP N-acyltransferase (OlsB) and acyl carrier protein (AcpP) from Pseudomonas putida or sequences substantially identical thereto. Another embodiment of the invention includes, without limitation, nucleic acid molecules encoding the aforementioned proteins that are substantially identical to the nucleotide sequences described herein.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the activity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Those skilled in the art will appreciate that polypeptides, peptides or peptide analogues can be synthesised using standard chemical techniques, for instance, by automated synthesis using solution or solid phase synthesis methodology. Automated peptide synthesisers are commercially available and use techniques known in the art. Polypeptides, peptides and peptide analogues can also be prepared from their corresponding nucleic acid molecules using recombinant DNA technology.

As used herein, the term “gene” refers to a nucleic acid that encodes a functional product, for instance an RNA, polypeptide or protein. A gene may include regulatory sequences upstream or downstream of the sequence encoding the functional product.

As used herein, the term “coding sequence” refers to a nucleic acid sequence that encodes a specific amino acid sequence. On the other hand, a “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, and translation leader sequences.

In some embodiments, the genes used in the method of the invention may be operably linked to other sequences. By “operably linked” is meant that the nucleic acid molecules encoding the ornithine acyl-ACP N-acyl transferase polypeptides and/or the acyl carrier protein of the invention and these regulatory sequences are connected in such a way as to permit expression of the proteins when the appropriate molecules are bound to the regulatory sequences. Such operably linked sequences may be contained in vectors or expression constructs which can be transformed or transfected into host cells for expression. It will be appreciated that any vector or vectors can be used for the purposes of expressing ornithine acyl-ACP N-acyl transferase and/or acyl carrier protein of the invention.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene in different cell types, or at different stages of development, or in response to different environmental or physiological conditions. A “constitutive promoter” is a promoter that direct the expression of a gene of interest in most host cell types most of the time. An “inducible promoter” is a promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters. In some embodiments of the present invention, one or more of the nucleic acids encoding the ornithine acyl-ACP N-acyltransferase or acyl carrier protein may be under the control of a strong constitute promoter. Several strong constitutive promoters are known in the art. Generally, strong constitutive promoters can deliver a high-level expression of a protein at all times in the microorganism, which is particularly useful for increasing the expression of a protein of interest. Such strong constitutive promoters include, but are not limited to Pcat, Pbla, dnak Promoter, htpG Promoter, PSF-OXB series in E. coli; PEM7, synthetic promoters (Köbbing et al., 2020) for P. putida; Pveg, PgsiB, PlepA, Pylb, PmreB, PspoM, PyknW, PsppA, PtrnQ, PsigX, PgroES for B. subtilis; P0208, P0627, P0019, P0407, P0392, P0230, P0785, or P0107, PGK1 promoter for Pichia pastoris and S. cerevisiae.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e., by human intervention. Recombinant nucleic acid constructs may be introduced into a host cell by transformation. Such recombinant nucleic acid constructs may include sequences derived from the same host cell species or from different host cell species.

As used herein, the term “chimeric”, means that a sequence comprises of sequences that have been “recombined”. By way of example sequences are recombined and are not found together in nature. The term “recombine” or “recombination” refers to any method of joining two or more polynucleotides. The term includes end to end joining, and insertion of one sequence into another. The term is intended to include physical joining techniques, for instance, sticky-end ligation and blunt-end ligation. Sequences may also be artificially synthesized to contain a recombined sequence. The term may also encompass the integration of one sequence into a second sequence by way of, for example, homologous recombination

The term “vector” refers to a means by which polynucleotides or gene sequences can be introduced into a cell. There are various types of vectors known in the art including plasmids, viruses, bacteriophages and cosmids. Generally, polynucleotides or gene sequences are introduced into a vector by means of a cassette. The term “cassette” refers to a polynucleotide or gene sequence that is expressed from a vector, for example, the polynucleotide or gene sequences encoding the acyl transferase polypeptides of the invention. A cassette generally comprises a gene sequence inserted into a vector, which in some embodiments, provides regulatory sequences for expressing the polynucleotide or gene sequences. In other embodiments, the vector provides the regulatory sequences for the expression of the acyl transferase polypeptides. In further embodiments, the vector provides some regulatory sequences, and the nucleotide or gene sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication.

The following examples are offered by way of illustration and not by way of limitation.

### Example 1

Cloning and Expression of the olsB Gene and the acpP Gene

The olsB gene (GenBank ID: MK430049.1) described in WO 2018/055563 A1, which encodes an ornithine acyl-ACP N-acyltransferase (OlsB), was cloned into the second multiple cloning site of the pETDuet-1 vector (Novagen catalogue ref. no. 71146-3) using the restriction enzyme sites NdeI and BglII.

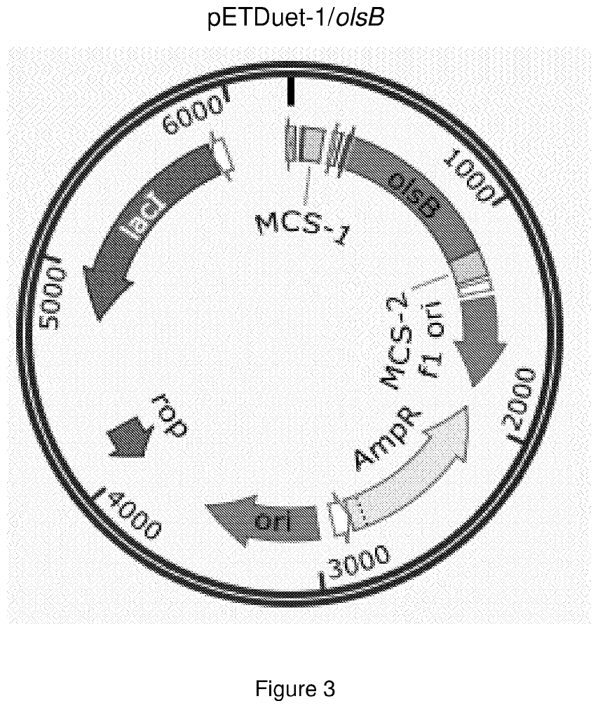

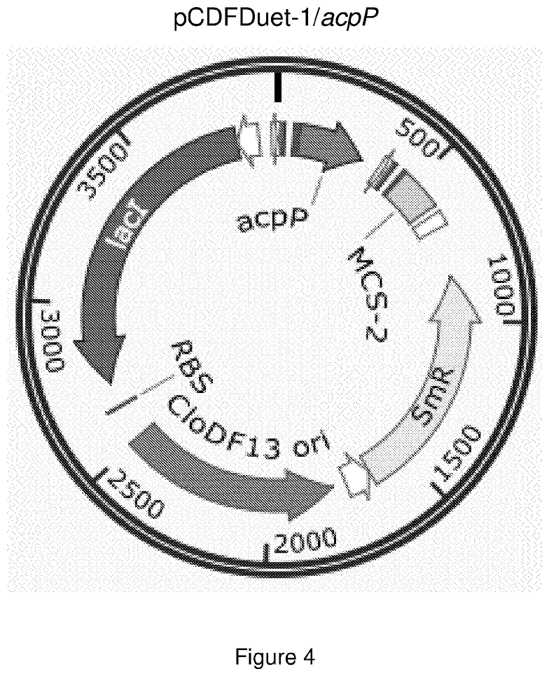

The acpP gene from Pseudomonas Putida KT2440 (GenBank ID: AE015451.2 locus 2158595-2158831) was cloned into the first multiple cloning site of pCDFDuet-1 (Novagen catalogue ref. no. 71340-3) using the restriction sites NcoI and NotI. The vectors pETDuet-1/olsB and pCDFDuet-1/acpP are shown in FIG. 3 and FIG. 4, respectively.

The resulting plasmids were transformed into a BL21 (DE3) E. coli cell line, genotype F-ompT hsdSB (rB-mB-) gal dcm (DE3) and were maintained by selection with ampicillin and streptomycin.

### Lyso-Ornithine Lipid Production and Biosurfactant Assay

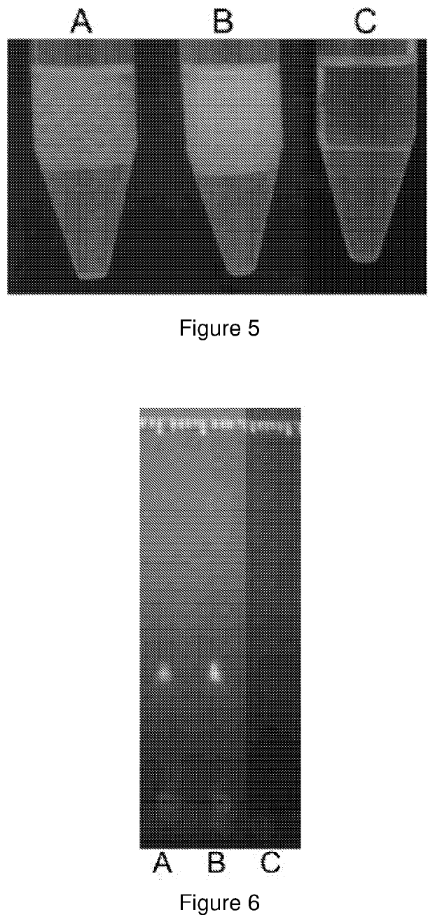

Cultures of the resulting cell line and control cell lines were grown at 37° C. with shaking at ~200 rpm in LB media supplemented with 20 mM ornithine. The cultures were grown to an optical density at 600 nm of 0.6 and the production of lyso-ornithine lipid (LOL) biosurfactant was induced by inducing expression of the two proteins with 1 mM IPTG for 24 hours at 37° C. with shaking at ~200 rpm. The cells were harvested by centrifugation (4000×g, 20 min, 4° C.) and the supernatants were recovered. The supernatants were tested for the presence of a biosurfactant by an emulsion assay where 2 ml of paraffin is mixed with 1 ml of supernatant and vortexed at full speed for 1 min after which they are incubated at room temperature for 24 hours and then inspected for emulsion. The result of this assay is shown in FIG. 5.

It was clear that expression of OlsB in cell line A was responsible for the production of the LOL biosurfactant as can be seen by the difference between the emulsion in cell line A and C, but the co-expression of the AcpP protein in cell line B led to a significantly thicker emulsion indicating the presence of higher amounts of biosurfactant. Cell line A, where only OlsB is expressed, has an emulsion with an E₂₄ value of 29.2, showing that the sole expression of OlsB leads to biosurfactant production. Cell line B has a thicker and larger emulsion than cell line A, with an E₂₄ value of 33.9. This indicates that the co-expression of OlsB with AcpP (cell line B) leads to higher production volumes of the biosurfactant.

Thin layer chromatography (TLC) was performed to visualize the difference in amounts of LOL production. The supernatants of the cultures were concentrated with a CentriVap concentrator (Labconco, Kansas City, USA) at a temperature of 42° C. to 10% of their initial volume. 20 cm×20 cm Silica gel 60 F254 aluminium backed TLC plates (Merck, Dramstadt, Germany) were cut into appropriate lengths with a constant height of 10 cm. Two microliters of the concentrated samples were applied onto the TLC plates, 0.5 μl at a time, drying with a hot air blower in between applications. The mobile phase consisted of chloroform/methanol/water mixture (64:35:4, v/v/v). The TLC plates were developed until the solvent front reached 1 cm from the top of the TLC plates. The plates were immediately dried with a hot air blower and stained with the fluorophore primuline (0.05% primuline, 80% acetone). Primuline's fluorescent emission increases in the presence of lipids and thereby identifies lipids on the TLC plates. The TLC plates were visualized under a UV lamp with a wavelength of 365 nm. The result of the TLC is shown in FIG. 6.

The results of the emulsion assay were confirmed and the increase in production of LOL was observed in the TLC in FIG. 2 as the brightness of the spot indicating the presence of LOL in the middle (B) of the TLC was significantly brighter.

### Example 2

### Lyso-Ornithine Lipid Production Optimisation and Quantification

The production of LOL by the produced cell line described in Example 1, cell line B, was further investigated and a method to quantify the production of LOL more accurately was developed using HPLC. HPLC was performed on a Dionex UltiMate 3000 HPLC (Thermo Fisher Scientific, Madison, USA) equipped with a Luna 5 μm C18 liquid chromatography column (250 mm×4.6 mm) (Phenomenex, Torrance, USA). The cleared culture supernatants were filtered through a 0.2 μm filter. A 200 μl sample of the supernatants was loaded onto the column. The flow rate was 1 ml/min and a gradient elution method was used [A: doubly distilled water; B: acetonitrile (Sigma, Saint Louis, USA)]: 40% B, 0 min; 40% B, 5 min; 70% B, 20 min; 95% B, 20.1 min; 95% B, 26 min; 40% B, 26.1 min; 40% B, 32 min. The temperature of the column was maintained at either 25° C. or 30° C. The absorbance at 200 nm was monitored. The peak areas were determined with the Chromeleon Dionex software version 6.8 (Thermo Fisher Scientific, Madison, USA).

Using the method described, the inventors were able to separate the LOL biosurfactant from all the other media components and thereby quantify the amounts of LOL. OlsB is known to produces lyso-ornithine lipids with different fatty acid carbon chain lengths, between 14 and 19 as was previously shown. Using the new method, had the unexpected result of separating the different LOLs from each other and not just from the media. This enables quantification of the amount of every type of LOL in the sample.

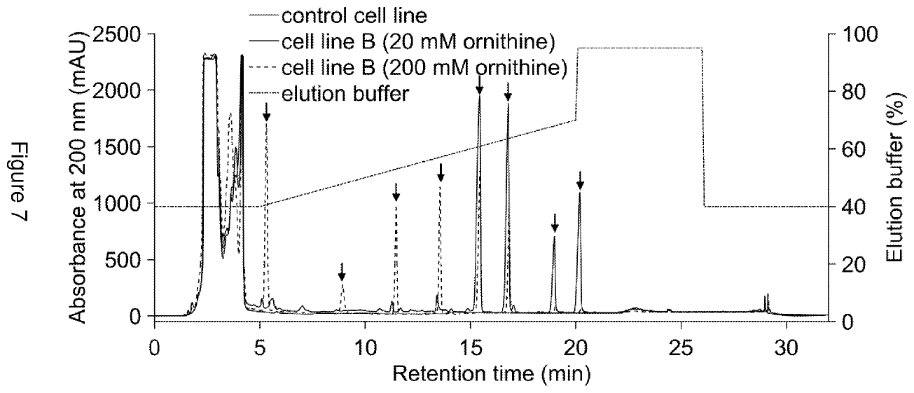

With reference to FIG. 7, the initial bulk peak between 0 and 5 minutes is attributed to the media. Peaks 9, 10, 12, 13, 15, and 16 were in the range where the inventors expected LOLs to appear. It was hypothesized that these regularly separated peaks could be attributed to the different fatty acid chain lengths of LOL. It was subsequently found that, if the conditions of the production are changed, certain peaks appear, and others disappear changing the composition of the LOLs. Mass spectrometry was performed, and the masses of the peaks were identified and confirmed to be different chain lengths of LOL shown in Table 1.

TABLE 1
Lyso-ornithine lipids identified in the HPLC separation technique.

| m/z [M + H]+ | Molecular formula | Lyso-ornithine lipid (LOL) |
|---|---|---|
| 303.230 | C15 H31 O4 N2 | C10:0 |
| 329.244 | C17 H33 O4 N2 | C12:1 |
| 331.260 | C17 H35 O4 N2 | C12:0 |
| 357.275 | C19 H37 O4 N2 | C14:1 |
| 359.29043 | C19 H39 O4 N2 | C14:0 |
| 385.30608 | C21 H41 O4 N2 | C16:1 |
| 387.32173 | C21 H43 O4 N2 | C16:0 |
| 413.33738 | C23 H45 O4 N2 | C18:1 |
| 427.35196 | C24 H45 O4 N2 | C19:1 |

The significance of this method was emphasised when the inventors found that the type of LOL produced, i.e., the length of their fatty acid chain, could be manipulated by changing the amount of ornithine supplementation. An ornithine concentration of 20 mM produced long-chain fatty acids and an ornithine concentration of 200 mM produced shorter-chain fatty acids (FIG. 7). Shorter and longer fatty acids have a significant effect on the application of the LOLs as a biosurfactant and the ability to manipulate the lengths that are produced could be of significant commercial value. This developed method was also used to screen for optimal biosurfactant-producing conditions with the traditional biosurfactant optimization methods.

A comparison between the LOL production of cell line A, the control cell line containing pETDuet-1/olsB and an empty pCDFDuet-1 vector at the standard conditions, and cell line B, the cell line containing pETDuet-1/olsB and pCDFDuet-1/acpP, at optimized conditions (media at pH 8, incubation temperature of 25° C.), was performed. The standard conditions were as follows: the E. coli BL21 (DE3) glycerol stocks containing the co-expression plasmids were used to inoculate 5 ml M9 minimal media containing 50 μg/ml ampicillin and streptomycin and incubated overnight (37° C., ~125 rpm). M9 minimal media (15-20 ml) containing 50 μg/ml ampicillin and streptomycin and 20 mM ornithine was inoculated with 1 ml of the overnight culture. The culture was incubated (37° C., ~125 rpm) until an OD₆₀₀ of ~0.6 was observed. Expression was induced by the addition of 1 mM IPTG. The cultures were incubated (37° C., ~125 rpm) for 24 hours. The cells were harvested by centrifugation (4000×g, 20 minutes, 4° C.) and the supernatants were recovered. The supernatants were filtered through a 0.20 μm filter and stored at 4° C.

Single culturing conditions were varied to identify conditions that produced higher amounts of lyso-ornithine lipid. The standard conditions were applied and either the temperature (25° C., 30° C., and 37° C.), media pH (pH 6, 7, and 8), ornithine supplementation (2, 20, and 200 mM) or IPTG induction (0.1, 1, and 5 mM) was varied.

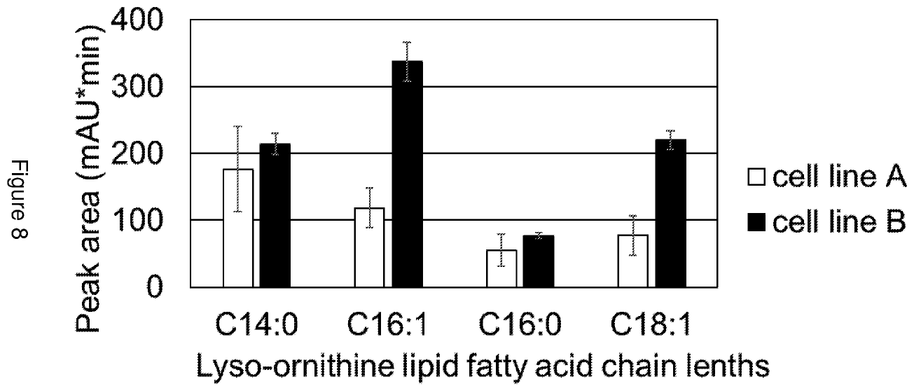

The HPLC quantification method was employed (column temperature at 30° C.) to determine what level of increase in LOL production is achieved with the co-expression cell line at more optimal conditions. This comparison was performed in triplicate and only the peaks areas of the main peaks produced at this ornithine supplementation were summed, i.e., LOLs with fatty acid chain lengths of C14:0, C16:1, C16:0 and C18:1. The ratio of the specific LOLs produced were determined (FIG. 8). The main difference between the LOL production of cell line A and cell line B is a prominent increase in LOLs containing fatty acids C16:1 and C18:1 in cell line B (FIG. 8). There is also an indication of an increase of LOLs containing fatty acids C14:0 and C16:0 in cell line B, but this increase is within the standard deviation of the two cell lines. Interestingly, the ratio of LOLs differs between cell line A and cell line B. In cell line A, there is more C14:0 LOL than C16:1 LOL. In cell line B, there is more C16:1 LOL than C14:0 LOL.

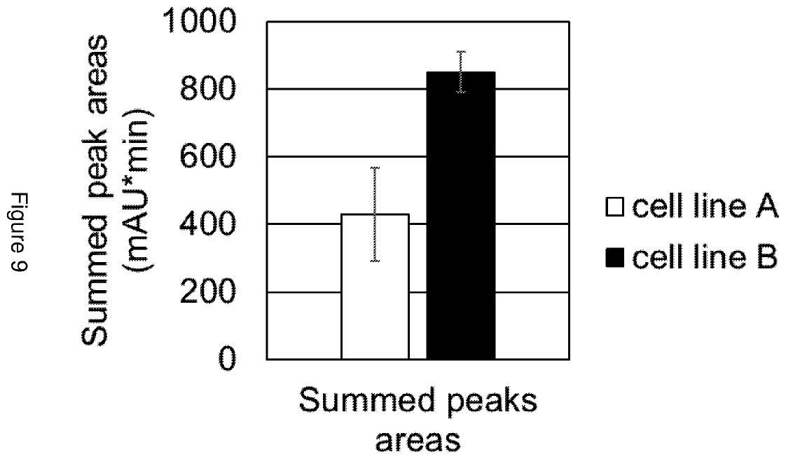

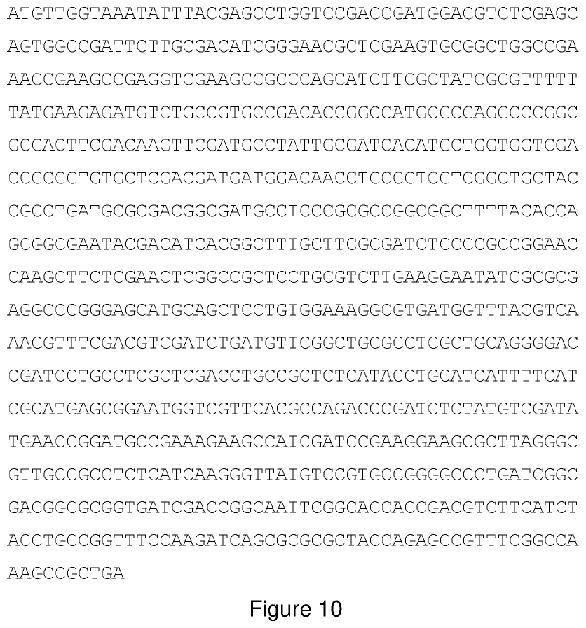

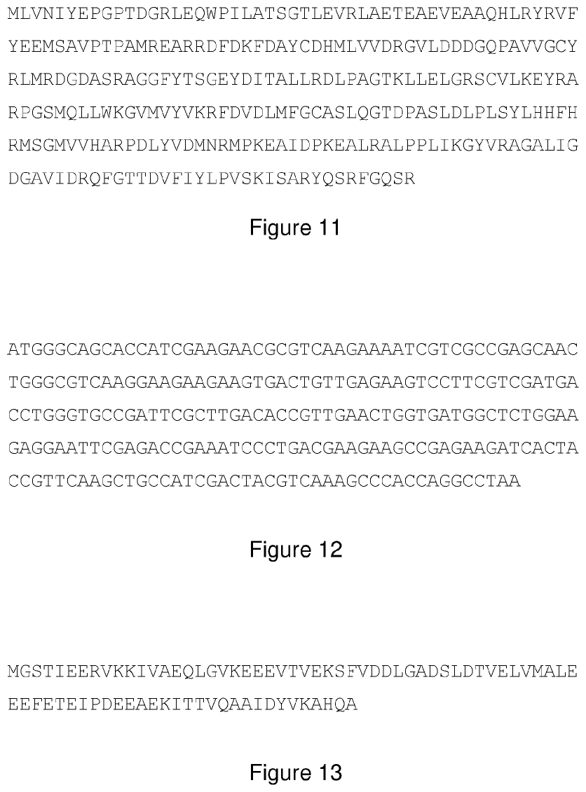

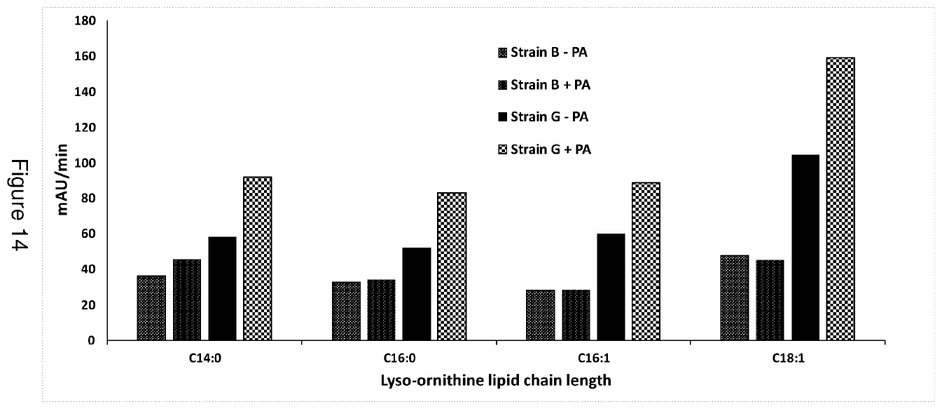

All the peak areas were summed (FIG. 9). The production of LOLs in cell line B at optimal conditions was 1.98 times more than cell line A at the standard condition (FIG. 9). This means that almost double the amount of LOLs are produced with the co-expression system at the optimized conditions than in the original strain at the standard conditions.

### Example 3

Co-Overexpression of the E. coli Pantothenate Kinase (Ec-panK) Along with Supplementation of the Fermentation Media with Panthothenic Acid (PA) to Increase Lyso-Ornithine Lipid (LOL) Synthesis in E. coli

Co-overexpression of the enzyme pantothenate kinase (panK or coaA) alongside olsB and acpP was investigated as a further mechanism for obtaining increased LOL production. In a previous study, it was observed that overexpression of heterologous or modified panK increased the intracellular coenzyme A (CoA) pool (Satoh et al., 2020; Kaku et al., 2022). As LOL is composed of two substrates (ornithine and a fatty acid), and as the inventors had already investigated the effect of supplementation of up to 200 mM ornithine, they reasoned that expression of panK may lead to a higher amount of available fatty acid which in turn might lead to increased LOL synthesis. However, the main benefit of an increased CoA pool through expression of Ec-panK [R106A] (Rock et al., 2003) for LOL synthesis was thought to be the ability of the recombinant strain to produce more holo-AcpP generated through the addition of a 4-phosphpantetheine group, donated by CoA, to apo-AcpP which is catalyzed by acyl carrier protein synthase (AcpS).

Expression constructs containing combinations of the metagenome-derived olsB, Pseudomonas putida-acpP and Escherichia coli-panK [R106A] (Rock et al., 2003) were created using standard cloning techniques and transformation of constructs into E. coli BL21-DE3 yielded the final expression strains (Table 2). Unless stated otherwise LOL production was assayed by culturing the strains in 30 ml M9 minimal medium at 25° C. supplemented with 1 mM IPTG and 20 mM L-ornithine in a 250 ml flask shaken at 150 rpm under the conditions indicated. Cultures were induced 6 hours after inoculation and cultured for 2-3 days following induction to allow for gene expression and compound synthesis. Culture supernatants were clarified through centrifugation at 3214×g for 10 min. Clarified supernatant was analyzed using a Thermo-Scientific Dionex Ultimate 3000 HPLC fitted with a Phenomenex Luna C18 (2) 250 mm×4.6 mm column (5 μm particle size). A flow rate of 1 ml·min⁻¹ was employed with the column kept at 40° C. and the mobile phase consisting of diH2O (A) and acetonitrile (B). 200 μl of sample was injected for analysis. The gradient consisted of: 5 min at 40% of B, linear increase from 40-70% of B over 15 min, 95% of B for 7 min followed by 5 min at 40% B. Compounds were detected at either 190 nm or 200 nm using a photo diode array and quantified at 200 nm to reduce contributions by baseline drift due to increasing acetonitrile concentrations.

To investigate LOL expression in the presence of overproduced pantothenate kinase (panK or coaA) four new constructs were engineered (expression constructs G, I, J and K in Table 2).

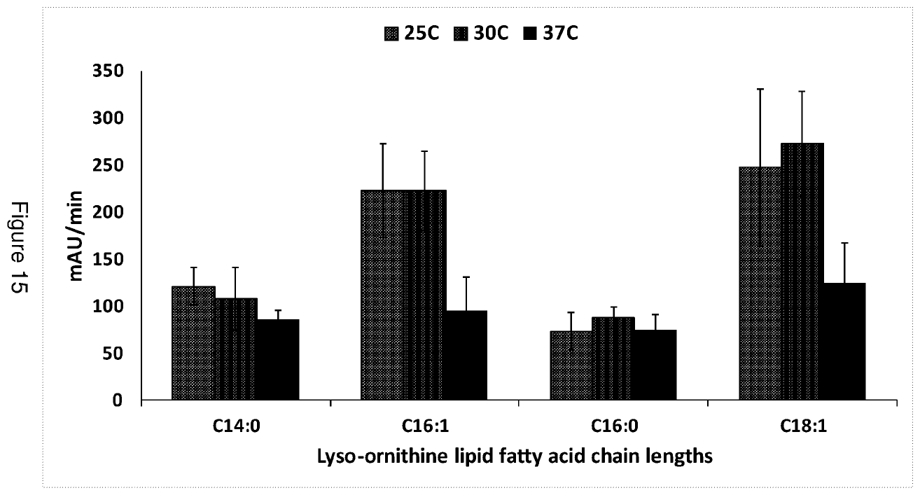

Following expression of the Ec-panK [R106A] alongside olsB and Pp-acpP in strain “G” and comparing it with the strain “B”, with reference to Table 2 below, it was evident that strains with panK [R106A] produced a greater quantity of LOL of each chain length than without the gene present also leading to greater overall LOL production (all peaks added together) (FIG. 14). The increase in overall LOL production resulting from the expression of panK [R106A] was 1.88-fold when considering the sum of all peak areas. Additionally, the inventors assayed whether supplementation of the medium with pantothenic acid (PA), the substrate for pank, would have a positive effect on LOL synthesis. Again, it was observed that addition of PA resulted in an even greater increase in LOL production for all four main products (FIG. 14). The cumulative increase of expression of panK [R106A] together with supplementation with pantothenic acid resulted in a 2.9-fold increase over the “B” strain fermented under standard conditions. This represents an additional 1.02-fold (2.9−1.88) increase over strains expressing pank only. Addition of PA to the “B” strain culture medium appeared to have a very small positive effect (1.05-fold increase) on overall LOL synthesis. This strongly suggest that the increase in LOL synthesis in strains expressing panK as well as the additional improvement in synthesis in the strains supplemented with pantothenic acid is directly attributable to expression of panK.

### Example 4

### Assessing the Effect of Different Growth Temperatures of LOL Production

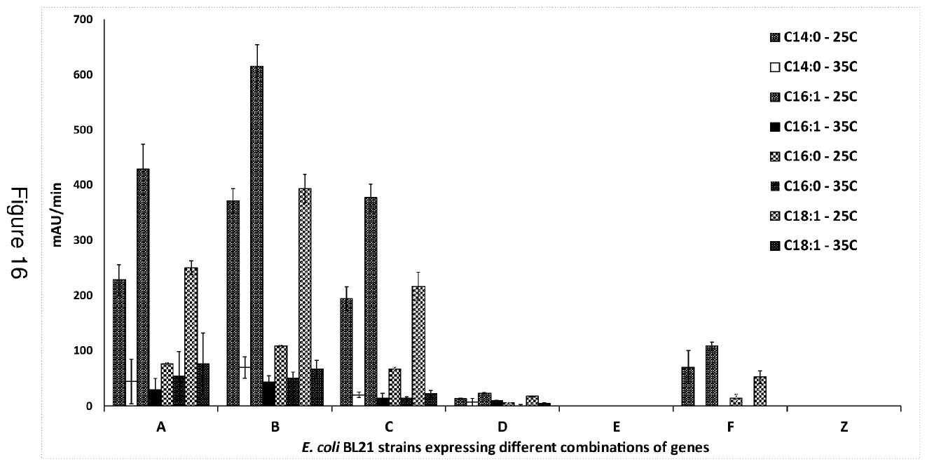

In an initial attempt to optimize the growth temperature for increased LOL production, the “B” strain, as detailed in Table 2 below, was used to assess the production of LOLs under different growth temperatures (FIG. 15). Although there was high variability in the data, it appears that there is a trend towards increased LOL production at lower temperatures with roughly equal production at 25° C. versus 30° C. and a marked decrease in production at 37° C. except for LOLs with C16:0.

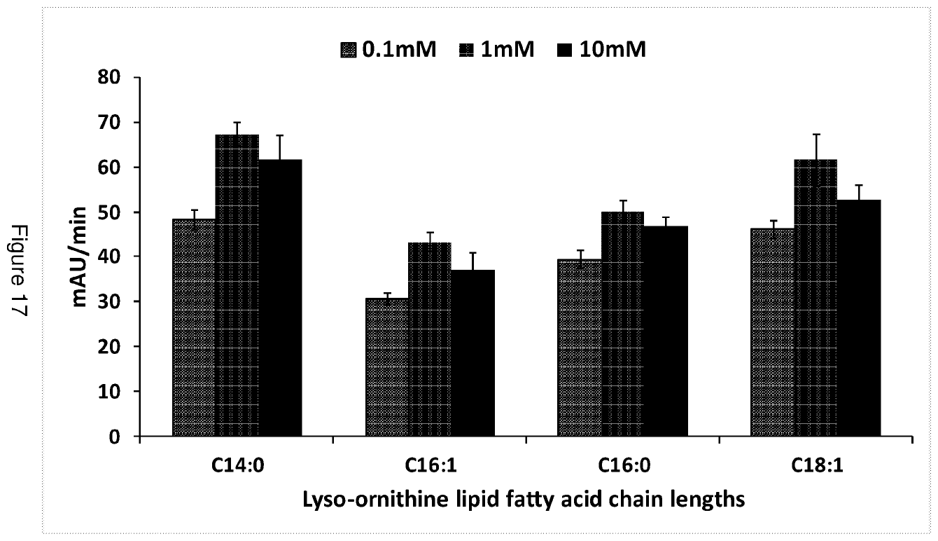

The inventors undertook a more comprehensive assessment, in which the “B” strain as well as strains “A”, “C”, “D”, “E”, “F” and “Z”, with reference to Table 2 below, were assessed for the production of LOLs at 25° C. and 35° C. (FIG. 16). The trend that was observed in the results presented in FIG. 15, was confirmed for the top three LOL producing strains “A”, “B” and “C” (FIG. 16). When comparing the total peak area for all four LOLs, co-overexpression of Pp-acpP with olsB (“B”-strain) resulted in a 1.5-fold increase over a strain expressing just olsB (“A” strain) at 25° C. but a 7.2-fold increase over the “A”-strain at 35° C. This demonstrates the significance of both the down shift in temperature and co-expression of Pp-acpP.

### Example 5

### Assessing the Effect of Different Concentrations of the Inducer IPTG on LOL Production

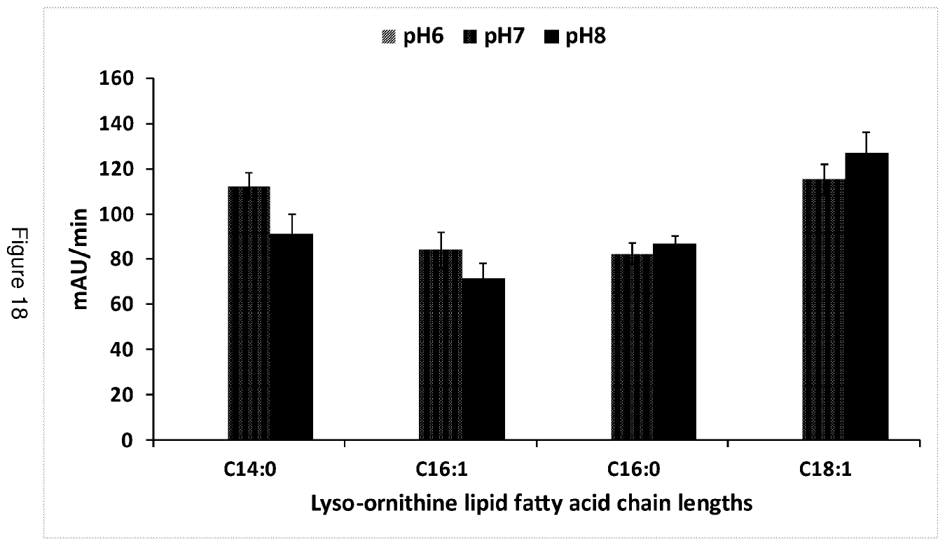

Isopropyl β-d-1-thiogalactopyranoside (IPTG) is used to induce gene expression. The effect of different concentrations of IPTG on LOL production in strain “B” is shown in FIG. 17. It was determined that the optimal inducer concentration is ~ 1 mM IPTG with a reduction in LOL production at 0.1 mM and 10 mM. This provides a good indication of the optimal IPTG concentration for possible further investigation to fine tune the inducer concentration, as the use of higher concentrations of IPTG represents a substantial proportion of the cost in producing these compounds. It is encouraging to observe ~74% of the LOL production (summed peak area for all four LOLs) at a 10-fold lower IPTG concentration than was expected.

### Example 6

### Assessing the Effect of the pH of the Growth Media on LOL Production

The effect of pH on LOL production in “B” strain was investigated, with the cell line being grown at pH 6, pH 7 and pH 8. As shown in FIG. 18, the pH of the growth media affects LOL production. Although growth media with pH 6 allowed the E. coli to grow, no LOL production was observed. It is possible that no LOL was produced or that the LOL was rapidly turned over under these conditions. Growth conditions at pH 7 or pH 8 gave the highest LOL yields, with slight variations in the ratios of the different chain lengths produced.

TABLE 2
Expression strains/cell lines generated in the study

| Expression | |
|---|---|
| Strain | Combination of genes and empty vectors present in the strain |
| A | olsB + pCDFDuet-1: Baseline control for the production of LOL by |
| | OlsB in the co-expression conditions. |
| B | olsB + acpP: To investigate LOL expression in the presence of |
| | overproduced AcpP. |
| C | olsB + acpP/acpS: To investigate LOL expression in the presence of |
| | overproduced AcpP and AcpS |
| D | olsB/aasS + acpP/acpS: To investigate LOL expression in the |
| | presence of overproduced AcpP, AcpS, and AasS. |
| E | aasS + acpP/acpS: Negative control to observe the background |
| | expression of LOL in the absence of OlsB. |
| F | olsB/aasS + pCDFDuet-1: To investigate LOL expression in the |
| | presence of overproduced AasS. |
| G | olsB + acpP + panK: To investigate LOL expression in the presence |
| | of overproduced AcpP and pantothenate kinase (panK or coaA) |
| I | olsB + acpP/acpS + panK: To investigate LOL expression in the |
| | presence of overproduced AcpP, AcpS and panK |
| J | olsB/aasS + acpP/acpS + panK: To investigate LOL expression in |
| | the presence of overproduced AcpP, AcpS, AasS and panK |
| K | olsB/aasS + pCDFDuet-1 + panK: To investigate LOL expression in |
| | the presence of overproduced AasS and panK |
| H | olsB + acpP + pRSFDuet-1: To investigate LOL expression in the |
| | presence of overproduced acpP. |
| X | pETDuet-1 + pCDFDuet-1 + pRSFDuet-1: Negative control to |
| | observe background expression of the cell line. |
| Z | pETDuet-1 + pCDFDuet-1: Negative control to observe background |
| | expression of the cell line. |

### REFERENCES

Kaku M, Ishidaira M, Satoh S, Ozaki M, Kohari D, Chohnan S. 2022. Fatty Acid Production by Enhanced Malonyl-CoA Supply in Escherichia coli. Curr. Microbiol. 79:269-264.
Köbbing S, Blank L M, Wierckx, N. 2020. Characterization of Context-Dependent Effects on Synthetic Promoters. Front. Bioeng. Biotechnol. 8:551.
Satoh S, Ozaki M, Matsumoto S. et al. 2020. Enhancement of fatty acid biosynthesis by exogenous acetyl-CoA carboxylase and pantothenate kinase in Escherichia coli. Biotechnol. Lett. 42:2595-2605.
Rock C O, Park H-W, Jackowski S. 2003. Role of feedback regulation of pantothenate kinase (CoaA) in control of coenzyme A levels in Escherichia coli. J Bacteriol. 185:3410-3415.

1. A method for increasing production of a lyso-ornithine lipid in a microorganism, the method comprising:
i. introducing into the microorganism a nucleic acid encoding a heterologous ornithine acyl-ACP N-acyltransferase, wherein the nucleic acid encoding the heterologous ornithine acyl-ACP N-acyltransferase is contained on an expression vector;
ii. expressing the heterologous ornithine acyl-ACP N-acyltransferase in the microorganism; and
iii. elevating levels of an acyl carrier protein in the microorganism;
wherein expressing the heterologous ornithine acyl-ACP N-acyltransferase catalyses the synthesis of lyso-ornithine lipid and elevating the levels of the acyl carrier protein increases the production of lyso-ornithine lipid in the microorganism.

2. The method of claim 1, wherein elevating levels of the acyl carrier protein in the microorganism comprises introducing into the microorganism a nucleic acid encoding the acyl carrier protein, optionally wherein the nucleic acid encoding the acyl carrier protein is operably linked to an inducible promoter or a strong constitutive promoter.

3. The method of claim 2, wherein the nucleic acid encoding the acyl carrier protein has a nucleotide sequence having at least 90% sequence identity with SEQ ID NO:4.

4. The method of claim 1, wherein the production of the lyso-ornithine lipid in the microorganism is increased relative to a microorganism that expresses the ornithine acyl-ACP N-acyltransferase and in which the levels of the acyl carrier protein have not been increased.

5. The method of claim 1, wherein the ornithine acyl-ACP N-acyltransferase has an amino acid sequence substantially identical to SEQ ID NO:1; and/or wherein the nucleic acid encoding the heterologous ornithine acyl-ACP N-acyltransferase has a nucleotide sequence having at least 90% sequence identity with SEQ ID NO:2.

6. The method of claim 1, wherein the acyl carrier protein is a Pseudomonas putida acyl carrier protein, optionally wherein the acyl carrier protein has an amino acid sequence substantially identical to SEQ ID NO:3.

7. The method of claim 1, wherein the acyl carrier protein is a heterologous acyl carrier protein.

8. The method of claim 1, wherein the microorganism is Escherichia coli or Pseudomonas putida.

9. The method of claim 1, wherein the method further comprises a step of culturing the microorganism in a medium containing ornithine, optionally wherein the concentration of ornithine in the medium is from about 2 mM to about 200 mM.

10. The method of claim 9, wherein changing the concentration of ornithine in the medium alters the length of the fatty acid chain of the lyso-ornithine lipid.

11. The method of claim 10, wherein a lower concentration of ornithine in the medium results in lyso-ornithine lipids with a relatively longer fatty acid chain and a higher concentration of ornithine in the medium results in lyso-ornithine lipids with a relatively shorter fatty acid chain.

12. The method of claim 1, wherein the method is carried out at a temperature of less than 35° C., optionally wherein the method is carried out at a temperature of about 25° C.

13. The method of claim 1, wherein the method is carried out in the presence of IPTG.

14. The method of claim 1, wherein the method is carried out at a pH of between about pH 7 and about pH 8.

15. The method of claim 1, wherein the method further comprises a step of co-expressing a pantothenate kinase enzyme in the microorganism.

16. The method of claim 1, wherein the method further comprises preparing a biosurfactant using the microorganism.

17. A recombinant microorganism capable of expressing elevated levels of a lyso-ornithine lipid, comprising:
i. a first nucleic acid encoding a heterologous ornithine acyl-ACP N-acyltransferase; and
ii. a second nucleic acid encoding an acyl carrier protein operably linked to an inducible promoter or a strong constitutive promoter;
wherein the first and second nucleic acids are contained on at least one expression vector and wherein expressing the heterologous ornithine acyl-ACP N-acyltransferase catalyses the synthesis of lyso-ornithine lipid and expressing the acyl carrier protein increases the production of lyso-ornithine lipid.

18. The recombinant microorganism of claim 17, wherein the production of the lyso-ornithine lipid in the microorganism is increased relative to a microorganism that only comprises a nucleic acid encoding the ornithine acyl-ACP N-acyltransferase and not the nucleic acid encoding the acyl carrier protein operably linked to the inducible promoter or the strong constitutive promoter.

19. The recombinant microorganism of claim 17, wherein the ornithine acyl-ACP N-acyltransferase has an amino acid sequence substantially identical to SEQ ID NO:1; and/or wherein the first nucleic acid has a nucleotide sequence having at least 90% sequence identity with SEQ ID NO: 2.

20. The recombinant microorganism of claim 17, wherein the acyl carrier protein is a Pseudomonas putida acyl carrier protein, optionally wherein the acyl carrier protein has an amino acid sequence substantially identical to SEQ ID NO:3; and/or wherein the second nucleic acid has a nucleotide sequence having at least 90% sequence identity with SEQ ID NO:4.

21. The recombinant microorganism of claim 19, wherein the acyl carrier protein is a heterologous acyl carrier protein.

22. The recombinant microorganism of claim 17, wherein the microorganism further comprises a third nucleic acid encoding a pantothenate kinase enzyme.

23. The recombinant microorganism of claim 17, wherein the recombinant microorganism is Escherichia coli or Pseudomonas putida.

24. A lyso-ornithine lipid produced by the method of claim 1.

25. Use of the lyso-ornithine lipid of claim 24 as a biosurfactant.


SEQUENCE LISTING (6 sequences)

SEQ ID NO: 1 - DNA, 843 bp, Pseudomonas putida
  source 1..843: mol_type = unassigned DNA
atgttggtaaatatttacgagcctggtccgaccgatggacgtctcgagcagtggccgattcttgcgacatcgggaacgctcgaagtgcggctggccgaaaccgaagccgaggtcgaagccgcccagcatcttcgctatcgcgttttttatgaagagatgtctgccgtgccgacaccggccatgcgcgaggcccggcgcgacttcgacaagttcgatgcctattgcgatcacatgctggtggtcgaccgcggtgtgctcgacgatgatggacaacctgccgtcgtcggctgctaccgcctgatgcgcgacggcgatgcctcccgcgccggcggcttttacaccagcggcgaatacgacatcacggctttgcttcgcgatctccccgccggaaccaagcttctcgaactcggccgctcctgcgtcttgaaggaatatcgcgcgaggcccgggagcatgcagctcctgtggaaaggcgtgatggtttacgtcaaacgtttcgacgtcgatctgatgttcggctgcgcctcgctgcaggggaccgatcctgcctcgctcgacctgccgctctcatacctgcatcattttcatcgcatgagcggaatggtcgttcacgccagacccgatctctatgtcgatatgaaccggatgccgaaagaagccatcgatccgaaggaagcgcttagggcgttgccgcctctcatcaagggttatgtccgtgccggggccctgatcggcgacggcgcggtgatcgaccggcaattcggcaccaccgacgtcttcatctacctgccggtttccaagatcagcgcgcgctaccagagccgtttcggccaaagccgctga

SEQ ID NO: 2 - protein, 280 aa, Pseudomonas putida
MLVNIYEPGPTDGRLEQWPILATSGTLEVRLAETEAEVEAAQHLRYRVFYEEMSAVPTPAMREARRDFDKFDAYCDHMLVVDRGVLDDDGQPAVVGCYRLMRDGDASRAGGFYTSGEYDITALLRDLPAGTKLLELGRSCVLKEYRARPGSMQLLWKGVMVYVKRFDVDLMFGCASLQGTDPASLDLPLSYLHHFHRMSGMVVHARPDLYVDMNRMPKEAIDPKEALRALPPLIKGYVRAGALIGDGAVIDRQFGTTDVFIYLPVSKISARYQSRFGQSR

SEQ ID NO: 3 - DNA, 240 bp, Pseudomonas putida
  source 1..240: mol_type = unassigned DNA
atgggcagcaccatcgaagaacgcgtcaagaaaatcgtcgccgagcaactgggcgtcaaggaagaagaagtgactgttgagaagtccttcgtcgatgacctgggtgccgattcgcttgacaccgttgaactggtgatggctctggaagaggaattcgagaccgaaatccctgacgaagaagccgagaagatcactaccgttcaagctgccatcgactacgtcaaagcccaccaggcctaa

SEQ ID NO: 4 - protein, 79 aa, Pseudomonas putida
MGSTIEERVKKIVAEQLGVKEEEVTVEKSFVDDLGADSLDTVELVMALEEEFETEIPDEEAEKITTVQAAIDYVKAHQA

SEQ ID NO: 5 - DNA, 957 bp, Escherichia coli
  source 1..957: mol_type = other DNA
atgcttatgagtataaaagagcaaacgttaatgacgccttacctacagtttgaccgcaaccagtgggcagctctgcgtgattccgtacctatgacgttatcggaagatgagatcgcccgtctcaaaggtattaatgaagatctctcgttagaagaagttgccgagatctatttacctttgtcacgtttgctgaacttctatataagctcgaatctgcgccgtcaggcagttctggaacagtttcttggtactaacgggcaacgcattccttacattatcagtattgctggcagtgtcgcggtggggaaaagtacaaccgccgctgtattgcaggcgctattaagccgttggccggaacatcgtcgtgttgaactgatcactacagatggcttccttcaccctaatcaggttctgaaagaacgtggtctgatgaagaagaaaggcttcccggaatcgtatgatatgcatcgcctggtgaagtttgtttccgatctcaaatccggcgtgccaaacgttacagcccctgtttactcgcatcttatttatgatgtgatcccggatggagataaaacggttgttcagcctgatattttaattcttgaagggttaaatgtcttacagagcgggatggattatccacacgatccacatcatgtatttgtttctgattttgtcgatttttcgatatatgttgatgcaccggaagacttacttcagacgtggtatatcaaccgttttctgaaattccgcgaaggggcttttaccgacccggattcctattttcataactacgcgaaattaactaaagaagaagcgattaagactgccatgacattgtggaaagagatcaactggctgaacttaaagcaaaatattctacctactcgtgagcgcgccagtttaatcctgacgaaaagtgctaatcatgcggtagaagaggtcagactacgcaaataa

SEQ ID NO: 6 - protein, 318 aa, Escherichia coli
MLMSIKEQTLMTPYLQFDRNQWAALRDSVPMTLSEDEIARLKGINEDLSLEEVAEIYLPLSRLLNFYISSNLRRQAVLEQFLGTNGQRIPYIISIAGSVAVGKSTTAAVLQALLSRWPEHRRVELITTDGFLHPNQVLKERGLMKKKGFPESYDMHRLVKFVSDLKSGVPNVTAPVYSHLIYDVIPDGDKTVVQPDILILEGLNVLQSGMDYPHDPHHVFVSDFVDFSIYVDAPEDLLQTWYINRFLKFREGAFTDPDSYFHNYAKLTKEEAIKTAMTLWKEINWLNLKQNILPTRERASLILTKSANHAVEEVRLRK